(19) United States
(12) Patent Application Publication
(10) Pub. No.: US 2011/0160696 A1
(43) Pub. Date: Jun. 30, 2011

(54) INJECTION PORT DEVICE ADAPTED FOR USE WITH INSULIN PUMP

(75) Inventor: Udo Hoss, Castro Valley, CA (US)

(73) Assignee: ABBOTT DIABETES CARE INC., Alameda, CA (US)

(21) Appl. No.: 12/982,781

(22) Filed: Dec. 30, 2010

Related U.S. Application Data
(60) Provisional application No. 61/291,573, filed on Dec. 31, 2009.

(51) Int. Cl. A61M 5/00

(52) U.S. Cl. 604/506; 604/288.01

(57) ABSTRACT

An injection port comprises a body having an outlet delivery cannula configured to pierce subcutaneous tissue of a patient to deliver medication. The port includes multiple injection sites, each of which can be used multiple times, and each of which is sealed. Each injection port is configured to temporarily receive a delivery device and reseal itself when the delivery device is removed. In one embodiment, a seal includes a pierceable stationary septum that reseals itself when a sharp cannula is removed. One injection site also includes locking features to receive a locking device of a delivery device and temporarily lock the delivery device in place at the injection site. The locking device may later be unlocked when delivery is complete and removed. In one application, a diabetic may use one injection site for syringes during the day and the locking injection site for a insulin pump during sleeping hours.

### CROSS-REFERENCES TO RELATED APPLICATIONS

This application claims the benefit of U.S. Application No. 61/291,573, filed Dec. 31, 2009, which is incorporated herein by reference in its entirety.

### BACKGROUND

The invention is generally directed to the delivery of medication and, more particularly, to a system and associated method for delivery of a medication through a single subcutaneous injection port by use of a sharpened cannula in one instance and by a pump in a second instance.

Insulin is used to control blood sugar in people who have type 1 diabetes (condition in which the body does not make insulin and therefore cannot control the amount of sugar in the blood) and in people who have type 2 diabetes (condition in which the blood sugar is too high because the body does not produce or use insulin normally). Insulin helps keep blood glucose levels on target by moving glucose from the blood into the body's cells. The cells then use glucose for energy. In people who don't have diabetes, the body makes the right amount of insulin on its own, but the bodies of diabetics do not.

Insulin is in a class of medications called hormones. There are different types of insulin but they differ only in how quickly they begin to work and how long they continue to control blood sugar or glucose. Insulin is usually needed several times a day, and more than one type of insulin may be needed. Insulin controls high blood sugar but does not cure diabetes.

The difficulty with the delivery of insulin is that it is a polypeptide and peptides are easily destroyed under the influence of proteolytic enzymes in the stomach and small intestines. Orally delivered insulin will not “live” long enough to have a beneficial effect since it will be destroyed by the stomach and intestinal enzymes before it can traverse the walls of the intestine. Scientists have tried a variety of means including insulin plasters, inhalations, capsules covered with a special protective coating, but none of these has ensured the required effect. Additives that make it easier for the intestine to absorb large molecules like insulin have been tried but have met with years of setbacks. Recent results are more favorable and signs of success may be at hand. Work continues on the development of an oral insulin delivery but for now, the vast majority of diabetics must use injection to deliver insulin to their bodies.

Injections are so uncomfortable and inconvenient that some patients are reluctant to use insulin frequently enough to adequately control their blood sugar. The insertion of a cannula, catheter, or other device through the skin for the delivery of an intravenous or subcutaneous medication can be difficult for many patients. All patients would prefer to avoid skin punctures but if complete avoidance is not possible, they would prefer to minimize the number of such insertions. Presently, the options available to insulin-dependent diabetics for insulin delivery include direct injections with a sharp cannula fitted to a syringe (referred to herein for convenience as a “syringe”) and continuous-delivery with an insulin pump. Each of these insulin delivery options has advantages and disadvantages. One of the main concerns of a patient, either consciously or subconsciously, is the pain and bruising resulting from skin punctures caused by such insulin deliveries.

One of the most reliable methods of insulin delivery that a diabetic can choose is direct injection with a syringe (i.e., a sharp injection cannula fitted to a syringe pierces the skin to deliver the medication subcutaneously). Direct injection with a syringe offers precise measurement of insulin and the security of manual delivery. However, direct injection with a syringe sometimes necessitates multiple injections during the course of a day. For example, a Type-1 diabetic generally needs a dosage of insulin either immediately before or after every meal. In some cases, such diabetics need insulin delivery during the course of sleeping hours.

For many diabetics, their aversion to cannula punctures precludes them from ever being able to bring themselves to direct injection with a syringe. For some who are able to inject themselves, the pain of multiple direct injections per day and the bruising at the injection site become difficult to tolerate. Regardless of the particular reason or reasons, there are a large number of diabetics who have difficulty observing a prescribed injection schedule due to their aversion to cannula punctures. Failure to inject medication when needed can result in under-medication which can endanger a diabetic's health. However, lessening the number of skin punctures per day while still delivering the needed quantity of medication can assist a patient in controlling his or her aversion to punctures.

The insulin pump has become an option for some diabetics who cannot face multiple daily direct injections with a syringe. With the insulin pump, a diabetic receives a continuous dosage of insulin from a pump apparatus via an “injection device” mounted on his or her body. Insulin is supplied (e.g., pumped) from the insulin pump through a tube to the injection device. Injection devices generally include a delivery cannula mounted in a subcutaneous manner through the skin of the diabetic. The injection device includes a channel that transmits insulin from an inlet port to the delivery cannula which results in delivery to the subcutaneous tissue layer of the diabetic in which the delivery cannula is located.

The use of an insulin pump also requires a puncture of the patient's skin. Mounting the injection device generally involves the use of an “insertion cannula.” Most conventional injection devices have an insertion cannula that extends through the body of the injection device and through the delivery cannula of the injection device. During mounting of such a conventional injection device, the insertion cannula serves to pierce the skin and to support the delivery cannula while being inserted, as most delivery cannulae are made from a soft and/or flexible material. Accordingly, the diabetic still must deal with a cannula piecing his or her skin. However, because the injection device may remain in place for an extended period of time (e.g., up to three days, or more), the diabetic need only deal with one injection-type cannula over those three or more days, rather than multiple times per day. This extended period of time between cannula insertions is what makes the pump tolerable for many diabetics who have an aversion to being punctured.

The insulin pump has disadvantages also. Two of those disadvantages are the size and weight of the pump and the cost. Finding an inconspicuous yet convenient place to wear the pump can be difficult, and the cost can be substantial. There are those patients who have an active lifestyle, who do not desire to continuously wear an insulin pump, and who are able to inject themselves with insulin by means of syringes. In the case of these patients, lowering the number of punctures would nevertheless be desirable. If such patients also have a need for insulin during sleeping hours, and awakening during the night for injections is not desirable, the use of an insulin pump during sleeping hours would help. Thus, such patients could inject themselves during the day with individual syringes requiring separate and multiple punctures, and then rely on the pump at night, which requires only one puncture for the entire night, and they can remain asleep. In any case, whether through direct injection or through use of an insulin pump, multiple punctures of the diabetic's skin is required.

Hence, those knowledgeable in this area have recognized a need for a system and method that enables active patients to inject themselves during their busy days as necessary and permits them to use an insulin pump during sleeping hours while at the same time, lessening the number of punctures these patients need to make of themselves. The present invention fulfills this need and others.

### SUMMARY OF THE INVENTION

Briefly and in general terms, the invention is directed to an injection port attached in place for an extended period having multiple injection sites each of which may be used multiple times by multiple delivery devices. In a first aspect, an injection port for delivery of medication through skin, the port comprises a body having a top and a bottom surface, an outlet cannula extending outwardly from the bottom surface of the body, the outlet cannula adapted to puncture skin of a patient, the outlet cannula having an inner surface that defines a cannula passageway through which medication is delivered, multiple injection sites formed in the body, each of which includes a channel connecting with the cannula passageway and each of which is configured to temporarily receive medication delivery devices multiple times for delivery of medication through the cannula passageway, wherein each injection site includes a seal that is configured to be bypassed when a delivery device is received by the respective injection site and each of which reseals its respective injection site upon removal of a delivery device from the injection site, and wherein an injection site further includes an associated locking feature configured to temporarily engage a complementary locking mechanism on a delivery device to temporarily secure the delivery device in position at the injection site for delivery of medication to the injection site until the locking mechanism is disengaged from the locking features of the respective injection site and the delivery device is removed therefrom.

In more detailed aspects, the locking feature is configured so as to not interfere with the receipt of delivery devices at the associated locking-feature injection site that do not include a locking mechanism. The locking feature is configured so as to not interfere with the receipt of delivery devices at the associated locking-feature injection site that can be received by the other injection sites. A first injection site is located at a position that is ninety degrees from a second injection site. The first injection site is located at the top surface of the injection port and the second injection site is located at a side surface of the injection port. A first injection site comprises a first stop configured to limit a length of a delivery device that is received within the first injection site.

In other detailed aspects, the first injection site is configured to receive a first delivery device having a sharpened cannula and an enlarged portion, the seal of the first injection site being stationary, self sealing, and having an outside surface and an inside surface, the inside surface being located within the respective channel of the injection site, the seal having a length so that the sharpened cannula may pierce the seal and extend completely through the inside surface of the seal into the channel of the first injection site while the enlarged portion remains outside the first injection site, the first stop comprising an opening located about the outside surface of the seal, the opening having a size that is smaller than the enlarged portion of the injection device so that the length of penetration of the sharpened cannula of an injection device into the first channel is accordingly limited. Each of the injection sites comprises a stop configured to limit a length of penetration of a delivery device into the respective channel. The first injection site is configured with a length and a location of the first stop so that a tip of a cannula of a delivery device will be restricted from extending into the cannula passageway.

In yet further detailed features, the injection port further comprises an adhesive disposed at the bottom surface, the adhesive adapted to hold the injection port at a selected position on skin of a patient for an extended period so that multiple injections may be made through the port to the patient without having to separately puncture the patient for each injection.

In other aspects, the locking feature comprises a recess formed in the body at the associated injection site, the recess comprising a locking surface, the locking recess and locking surface configured to receive a locking mechanism having an arm with a locking barb, the arm being resiliently bendable to be introduced into and withdrawn from the recess, and the arm formed to be self-springing to a normal position when fully engaged with the recess so that the barb will engage the locking surface of the recess to thereby temporarily lock the mechanism into a fixed position in relation to the injection site. The locking feature comprises a plurality of recesses formed in the body on either side of the associated injection site, each recess comprising a locking surface, the locking recesses and associated locking surfaces configured to receive a complementary locking mechanism having a plurality of arms with each arm having a locking barb, and the recesses being spaced apart from each other so that the arms of the locking mechanism must resiliently bend inwardly to be introduced into and withdrawn from the recesses, and each arm having a barb to engage a locking surface of the respective recess in which the arm is placed to thereby temporarily lock the mechanism into a fixed position in relation to the associated injection site. The injection port further comprises a locking mechanism configured to be mounted at a distal end of a delivery device to engage the locking features of the body, wherein the locking features comprise a plurality of recesses formed in the body on either side of an associated injection site, each recess comprising an inner locking surface, the locking mechanism comprising a plurality of resiliently bendable locking arms with each arm having a locking barb formed for engaging a locking surface, each arm being resiliently bendable inwardly to be introduced into and withdrawn from a recess, and each arm having a normal configuration at which the barb engages the locking surface of the respective recess in which the arm is placed to thereby temporarily lock the mechanism into a fixed position in relation to the associated injection site, and each arm having an associated bend tab configured and located so that pressing the tabs toward each other causes the arms to bend inwardly to a position at which they may be introduced into or withdrawn from the recesses.

In additional more detailed aspects, there is provided an injection port for delivery of medication through skin, the port comprising a body having a top and a bottom surface, an outlet cannula extending outwardly from the bottom surface of the body, the outlet cannula adapted to puncture skin of a patient, the outlet cannula having an inner surface that defines a cannula passageway through which medication is delivered, first and second injection sites formed in the body, each of which includes a channel connecting with the cannula passageway and each of which is configured to temporarily receive medication delivery devices multiple times for delivery of medication through the cannula passageway, wherein each injection site includes a stationary seal that is configured to be pierceable by a sharp cannula of a delivery device when such delivery device is received by the respective injection site and each of which reseals itself and its respective injection site upon removal of the sharp cannula from the seal and the delivery device from the injection site, wherein the second injection site further includes a locking feature configured to temporarily engage a complementary locking mechanism on a delivery device to temporarily secure the delivery device in position at the second injection site for extended delivery of medication through the second injection site until the locking mechanism is disengaged from the locking feature of the second injection site and the delivery device is removed therefrom, wherein the locking feature is configured so as to not interfere with the receipt of delivery devices at the second injection site that can be received at the first injection site.

In accordance with method aspects, there is provided a of injecting a patient with medication comprising inserting a sealed injection port through the skin of a patient and attaching the inserted injection port to the skin to keep it in place for an extended period, temporarily engaging the injection port at a first sealed injection site thereon with a first delivery device to deliver medication to the patient through the first sealed injection port, removing the first delivery device after delivery of the medication and resealing the first injection site, temporarily engaging the injection port at a second sealed injection site thereon with a second delivery device to deliver medication to the patient through the second sealed injection port from a pump, locking the second delivery device to the second injection port so that the second delivery device will remain securely in operative position at the second injection site of the port for an extended period, unlocking the second delivery device from the second injection port and removing the second delivery device after delivery of the medication, and resealing the second injection site, wherein the steps of engaging, delivering, removing, and resealing may be performed multiple times over the extended period that the injection port is in place.

More detailed aspects include the step of engaging comprising piercing a stationary seal with a sharp cannula through which medication is delivered. The step of locking comprising bending a pair of locking arms toward each other, inserting the bent locking arms into recesses formed in the injection port, once within the recesses, allowing the arms to unbend in the recesses during which each arm engages a locking surface in the recess and resists pulling the arm from the recess thereby locking a delivery device to the injection port, and after delivery of the medication, bending the arms inward to release the arms from the locking surfaces, and pulling the arms from the recesses to unlock them.

Various features and advantages of the invention will become more apparent by the following detailed description of several embodiments thereof with reference to the attached drawings.

### BRIEF DESCRIPTION OF THE DRAWINGS

FIG. 1 is a side cross-sectional view of an injection port having an outlet cannula configured for insertion through a patient's skin to extend into the subcutaneous tissue for the delivery of medication, the port having a first injection site at the top for receiving the sharpened cannula of a syringe combination and a second injection site at the side for receiving a sharpened cannula connected to an insulin pump or other device, the figure also showing a pump cannula connector at the right side wherein the insulin pump cannula is mounted within a locking mechanism adapted to be removably connected and locked to a locking feature formed at the injection port, with both the first and the second injection sites of the port being sealed;

FIG. 2 is a top partial cross sectional view of FIG. 1 showing the insulin pump cannula and mounting mechanism securely locked into position at the second injection site of the injection port for extended delivery of insulin to the patient, the snap arms of the mounting mechanism having moved outward to engage locking surfaces of the locking feature of the injection site to hold the locking mechanism and pump cannula in operative position at the port for an extended period;

FIG. 3 is also a partial cross-sectional top view of FIG. 1 showing the disengagement of the locking arms of the locking mechanism and the partial removal of the locking mechanism and pump cannula from the locking feature of the injection site;

FIG. 4 is a cross-sectional side view of a second embodiment of an injection port also having two injection sites as in FIG. 1, wherein the first, top surface, injection site has space below its channel seal for delivery of medication from the sharpened syringe cannula without having the sharpened syringe cannula extend into contact with the patient's skin, as is shown, and the second, side injection site, has a sharpened insulin pump cannula fully engaged at the site with the mounting arm of a locking mechanism shown in dashed lines within the injection port body; and

FIG. 5 is a cross-sectional side view of FIG. 4 showing a syringe being used to deliver medication at the side injection site of the port rather than an insulin pump cannula, demonstrating that the locking feature of the injection site is configured such that other delivery devices may be used there.

### DETAILED DESCRIPTION OF THE PREFERRED EMBODIMENTS

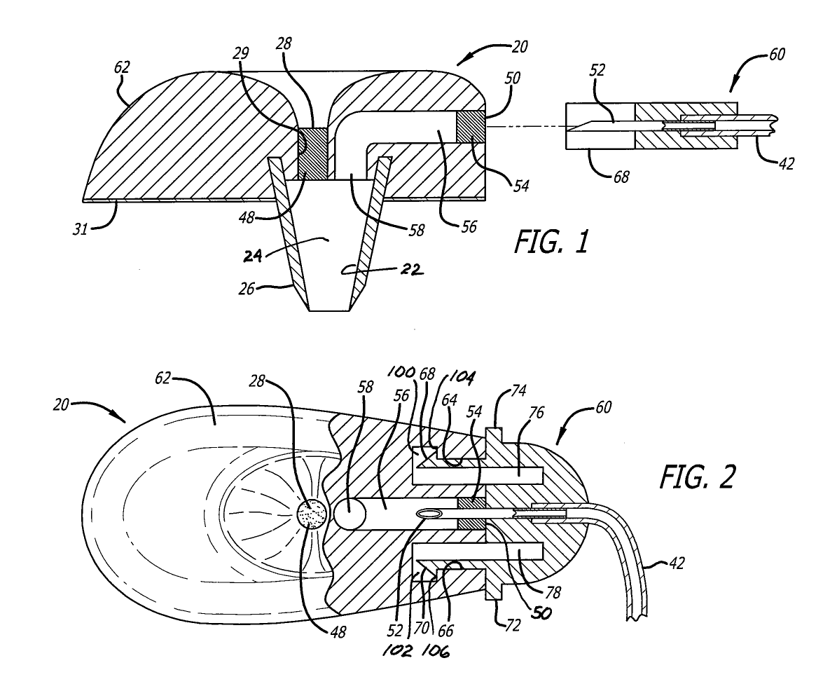

Turning now to the drawings with more particularity, wherein like reference numerals indicate like or similar elements among the views, there is shown in FIG. 1 an injection port 20 in accordance with aspects of the invention. The injection port 20 is designed to stay in place on a patient's skin over a period of time so that multiple deliveries of medication, such as insulin, can be given to the patient through the port without having to separately pierce the patient's skin multiple times. The injection port is inserted once through the patient's skin and leaves an effectively sealed outlet cannula 26 in place in the patient's subcutaneous tissue, which can be used repeatedly for injections. The outlet cannula 26 has an inner surface 22 that defines a cannula passageway 24 through which medication is delivered to the subcutaneous tissue.

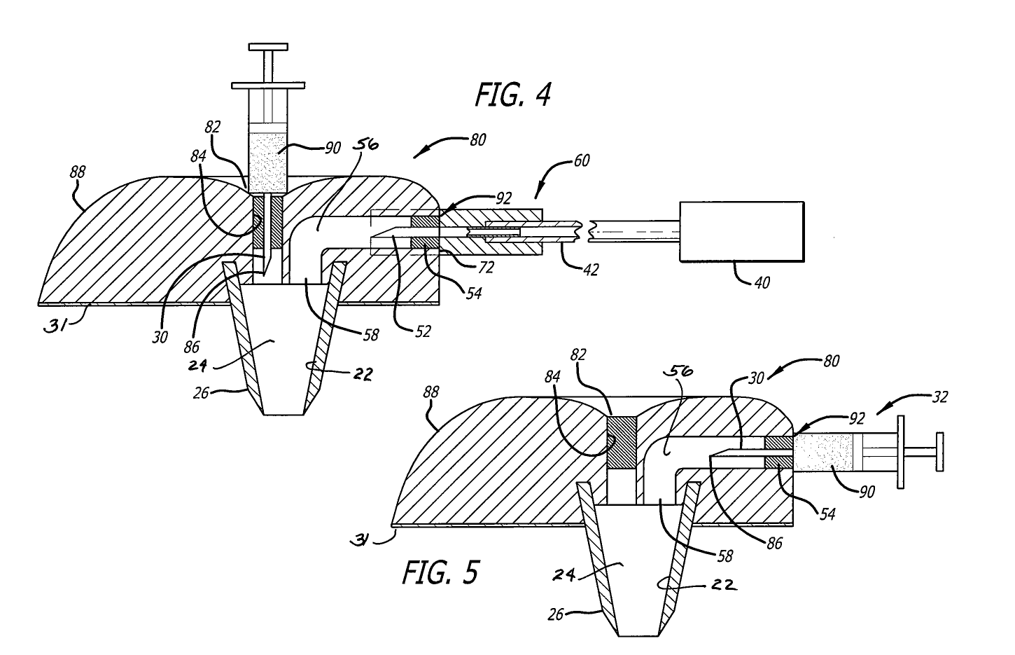

The injection port 20 includes a plurality of injection sites. A first injection site 28 is located at its top surface and is adapted to receive a sharpened cannula 30 of a syringe 32 (see the syringe shown in FIG. 4). For convenience, and not for limitation, this first site is referred to herein as a syringe site 28. The injection port 20 is also compatible with other medication delivery devices, such as an insulin pump 40 (see FIG. 4), so that the port 20 can be used for syringes, pen injectors, insulin pumps, and other delivery devices. As is described below, the design, configuration, and adaptation of the port 20 is such that multiple delivery devices can be used at the plurality of injection sites multiple times over the period of time that the port can remain on the patient.

A benefit of an injection port 20 in accordance with the invention is that patients can use a pump to deliver medication to themselves but can also change to individual injections at any time without requiring a new puncture through their skin, and vice versa. For example, if a patient uses a pump at night, but then during the day desires to switch to individual injections when needed, he or she can simple disconnect the pump from the port in the morning and use the injection sites during the day for individual syringe injections. Then at night, the patient again connects the pump. The self-sealing ports of the embodiments shown allow for such multiple connections and disconnections.

The components of FIG. 1 are not drawn to scale but instead may be exaggerated so that the embodiment will be better understood. The syringe injection site 28 is useful for syringes and pen injectors. It is sealed 48 with standard material and will reseal itself after a sharp syringe cannula is withdrawn from the seal 48. The injection port 20 also includes a second injection site 50 in this embodiment that is adapted to receive the sharpened cannula of an insulin pump 40 (FIG. 4). The second injection site 50 is likewise sealed with a resealable material 54. Both injection sites 28 and 50 have channels that connect with the cannula passageway 24. In the case of the first injection site 28, the channel 29 is fully filled with the seal 48 but in another embodiment, an open length may exist. Such a configuration is shown in FIG. 4. The second injection site 50 has a channel 56 that also opens 58 at the cannula passageway 24. Therefore, both sites are fluidly connected with the cannula passageway 24 by channels for the delivery of injected medication to the patient.

In the embodiments shown herein, the self-sealing members 48 and 54 that operate to seal their respective injection sites 28 and 50 are mounted within their respective channels 29 and 56 so that they are stationary. Each seal has an exposed outer surface to receive, in this embodiment, a sharpened cannula, and an inner surface that faces internally to the port 20. The seals 48 and 54 limit contaminants from entering the injection port 20 and limit the back-flow of medication and bodily fluids from the cannula passageway 24.

Also shown in FIG. 1, an insulin pump cannula 52 has a locking mechanism 60 for securing the pump cannula 52 in position at the second injection site 50. The operation of one embodiment of such a locking mechanism 60 can be more clearly seen by reference to the top partial cross-section view presented by FIG. 2. The body 62 of the injection port 20 includes a locking feature 36 associated with the second injection site 50. In this embodiment, the locking feature includes two recesses 64 and 66 that are formed in the body 62 to receive two barbed locking arms 68 and 70 of the locking mechanism 60 of the pump cannula 52. It can be noted that the outer surfaces of the arms are sized to just fit within the recesses, but that the recesses include two internal relieved areas 100 and 102 where the barbs are received. Those relieved areas each include a locking surface 104 and 106 with which the barb will engage to prevent the locking mechanism 60 from pulling out of the locking feature 36 of the body 62. By means of this locking system, the patient can connect an insulin pump to the injection port and be assured that the delivery tube of that pump will remain in communication with the patient's subcutaneous tissue for continuous delivery of the medication.

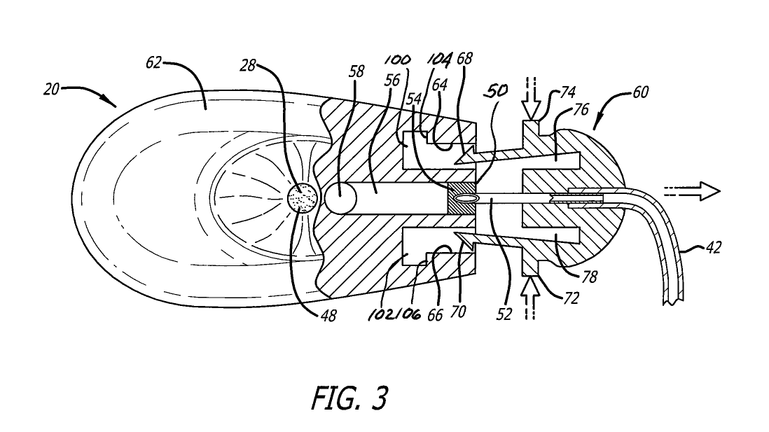

Another feature of the embodiment shown in FIGS. 2 and 3, is that the locking mechanism 60 can be easily disconnected from the locking feature 36 of the body 62. Side tabs 72 and 74 are provided at the base of the barbed locking arms 68 and 70 in this embodiment for assisting a user in bending the arms inwardly so that their barbs disengage from the locking surfaces 104 and 106 of the recesses 64 and 66. The locking mechanism 60 includes two relieved areas 76 and 78 that allow the arms 68 and 70 to be bent inward as shown by the arrows in dashed lines in FIG. 3. Once the arms have been bent inward enough and the barbs are disengaged from the locking surfaces a shown in FIG. 3, it is a simple matter to pull the arms of the locking mechanism 60 and pump cannula 52 free of the locking feature 74 and the injection port 50. Upon withdrawal of the cannula 52, the seal 54 automatically reseals itself.

As shown in FIG. 3, the sharpened cannula 52 and tube 42 from the insulin pump 40 (see FIG. 4) are embedded in the locking mechanism 60. This is only one embodiment of a locking mechanism. It is likely that others can be designed that will perform the same function. The invention is not intended to be limited by this particular embodiment of a locking mechanism.

FIG. 4 presents a different embodiment of an injection port 80 in accordance with aspects of the invention. In this embodiment, a syringe injection site or first injection site 82 has been modified from that shown in FIG. 1. In this case, the channel 84 used to receive the sharpened cannula 86 of a syringe 30 has been lengthened so that the sharpened point 86 of the syringe will not extend below the body 88 of the injection port 80 into the patient. Nevertheless, the medication 90 injected by the syringe will be deposited in the cannula passageway 24 and be conducted to subcutaneous tissue of the patient. In the case of both the first and second injection sites 82 and 92, a cannula stop may be used to limit the depth of penetration of the syringe or insulin pump cannula into the injection port 80. The port is configured in such a way that it contacts an enlarged portion of the syringe or cannula structure so that it controls the depth of the sharpened cannula into the port. In the case of the syringe 32, the syringe barrel is an enlarged portion and is made to be larger in diameter than the diameter of the opening 82 of the syringe injection site thus “stopping” the syringe from getting closer to the injection port 80 than the point of contact of the barrel with the upper surface of the injection port 80. Additionally, the length of the sharpened syringe cannula 30 is limited so that it remains within the body 88 of the injection port 80, as shown in FIG. 4 and does not protrude into the patient's skin. In the case of the insulin pump cannula 52, it includes a stop surface 72 at its base that contacts the side surface of the body 88 thereby “stopping” further penetration of the sharp cannula 52 into the body 88.

As in the other figures, components of the embodiment of FIG. 4 may not be drawn to scale. This is for purposes of clarity of illustration and it is not intended that the invention be limited by the relative sizes of the components as shown. Additionally, certain features may be changed yet still be within the bounds of the invention. For example, the figures show that the channel 56 of the insulin pump cannula proceeds sideways and then downwards to face the outlet cannula 26. In another embodiment, the insulin pump channel 56 may merge with the syringe injection site channel 84. Other configurations are possible. Additionally, the connection of the insulin pump 40 to the injection site 80 may employ different types of connectors, instead of only a sharpened cannula. Self-sealing blunt connectors may instead by used, as appropriate.

Both injection ports 20 and 80 of FIGS. 1 and 4 are compatible with connection to an insulin pump 40. It will also be noted from referring back to FIGS. 1 and 3 that the locking feature 36 of the body is internal and would not interfere with use of the second injection sites 54 and 92 respectively with other medication delivery devices. Indeed, FIG. 5 shows that the same syringe 32 used at the first injection site 82 in FIG. 4 can be used at the second injection site in FIG. 5. The second injection site 92 of the embodiment shown in the figures is different in that it includes a means to lock a delivery device in position so that use of the delivery device can be made over an extended period. This would assist the patient in obtaining the delivery of insulin during sleeping hours without fear that the delivery cannula may inadvertently pull out of the injection site of the port. In addition to sharpened cannula use at the second port 92, other types of delivery devices may be envisioned having locking mechanisms that will positively engage the locking feature 36 of the body to remain in delivery position for extended period. In addition to a catheter for use with an insulin pump, a patch pump may also be used.

The body of the injection port 62 and 88 may be attached to the patient by means available in the art today, such as with an adhesive 31 (FIG. 1). This adhesive may be applied to the bottom of the injection port or may exist on a separate sheet that is attached between the injection port and the patient. Other configurations are possible.

Although primarily described in the embodiments above as a sharpened cannula system, other connector types may be usable, depending on the application. For example, a needle-free connector system may be usable in which sharpened needles are not used and instead blunt cannulae and movable valve mechanisms are used. Such systems are shown in U.S. Pat. Nos. 5,676,346 and 5,685,866 and are incorporated herein by reference.

An injector port-type device in accordance with embodiments shown and described herein is mountable on a patient with the outlet cannula extending into or through subcutaneous tissue of a patient. Once the injection port is mounted on the patient, the patient may use syringe and pump injection cannulae through the existing injection port, instead of making new skin punctures each time medication delivery is needed. The patient is therefore spared having his or her skin punctured multiple times per day by injection cannulae. The patient would only be subjected to puncturing his or her skin by a cannula when replacing an existing mounted injection port with a new one. Depending on various factors, the patient may leave such an injection port in place for up to three days, and perhaps longer. The locking mechanism keeps the catheter in place at the injection port and will resist inadvertent patient movements that may tend to dislodge the catheter from the injection site.

An injection port in accordance with aspects of the invention reduces the number of punctures a patient must endure on a daily basis, and further provides a more versatile injection port in that a variety of syringes, pen injectors, and insulin pumps may be used with it. This device will provide a distinct benefit to patients who prefer injection by syringe but who need the delivery of medication by pump at other times. At night, an insulin pump may be connected to the injection port to deliver programmed amounts of insulin over the period of time that the patient sleeps. During an active day, the insulin pump may be disconnected and the patient may use the same injection port for the delivery of insulin by syringe or pen injectors. Due to these advantages, an injection port in accordance with the invention offers those patients who have an aversion to cannula punctures a much less invasive system while at the same time facilitating use of an insulin pump.

It is believed that such an injection port can be manufactured at a relatively low cost so that it can be made available to a wide variety of patients. It may also be made to have a relatively small size. The reduced anxiety and discomfort resulting from use of such an injection port, coupled with its lower cost, will provide patients with a significant benefit.

While the invention has been described in connection with what are presently considered to be the most practical and preferred embodiments, it is to be understood that the invention is not to be limited to the disclosed embodiments, components, and elements, but, to the contrary, is intended to cover various modifications, combinations of features, equivalent arrangements, and equivalent components and elements included within the spirit and scope of the appended claims. Thus, it is intended that the present invention covers modifications and variations of the examples shown.

What is claimed is:

1. An injection port for delivery of medication through skin, the port comprising:
a body having a top and a bottom surface;
an outlet cannula extending outwardly from the bottom surface of the body, the outlet cannula adapted to puncture skin of a patient, the outlet cannula having an inner surface that defines a cannula passageway through which medication is delivered;
multiple injection sites formed in the body, each of which includes a channel connecting with the cannula passageway and each of which is configured to temporarily receive medication delivery devices multiple times for delivery of medication through the cannula passageway;
wherein each injection site includes a seal that is configured to be bypassed when a delivery device is received by the respective injection site and each of which reseals its respective injection site upon removal of a delivery device from the injection site;
wherein an injection site further includes an associated locking feature configured to temporarily engage a complementary locking mechanism on a delivery device to temporarily secure the delivery device in position at the injection site for delivery of medication to the injection site until the locking mechanism is disengaged from the locking features of the respective injection site and the delivery device is removed therefrom.

2. The injection port of claim 1, wherein the locking feature is configured so as to not interfere with the receipt of delivery devices at the associated locking-feature injection site that do not include a locking mechanism.

3. The injection port of claim 1, wherein the locking feature is configured so as to not interfere with the receipt of delivery devices at the associated locking-feature injection site that can be received by the other injection sites.

4. The injection port of claim 1, wherein a first injection site is located at a position that is ninety degrees from a second injection site.

5. The injection port of claim 4, wherein the first injection site is located at the top surface of the injection port and the second injection site is located at a side surface of the injection port.

6. The injection port of claim 1, wherein a first injection site comprises a first stop configured to limit a length of a delivery device that is received within the first injection site.

7. The injection port of claim 6, wherein the first injection site is configured to receive a first delivery device having a sharpened cannula and an enlarged portion, the seal of the first injection site being stationary, self sealing, and having an outside surface and an inside surface, the inside surface being located within the respective channel of the injection site, the seal having a length so that the sharpened cannula may pierce the seal and extend completely through the inside surface of the seal into the channel of the first injection site while the enlarged portion remains outside the first injection site, the first stop comprising an opening located about the outside surface of the seal, the opening having a size that is smaller than the enlarged portion of the injection device so that the length of penetration of the sharpened cannula of an injection device into the first channel is accordingly limited.

8. The injection port of claim 6, wherein each of the injection sites comprises a stop configured to limit a length of penetration of a delivery device into the respective channel.

9. The injection port of claim 6 wherein the first injection site is configured with a length and a location of the first stop so that a tip of a cannula of a delivery device will be restricted from extending into the cannula passageway.

10. The injection port of claim 1, further comprising an adhesive disposed at the bottom surface, the adhesive adapted to hold the injection port at a selected position on skin of a patient for an extended period so that multiple injections may be made through the port to the patient without having to separately puncture the patient for each injection.

11. The injection port of claim 1, wherein the locking feature comprises a recess formed in the body at the associated injection site, the recess comprising a locking surface, the locking recess and locking surface configured to receive a locking mechanism having an arm with a locking barb, the arm being resiliently bendable to be introduced into and withdrawn from the recess, and the arm formed to be self-springing to a normal position when fully engaged with the recess so that the barb will engage the locking surface of the recess to thereby temporarily lock the mechanism into a fixed position in relation to the injection site.

12. The injection port of claim 1, wherein the locking feature comprises a plurality of recesses formed in the body on either side of the associated injection site, each recess comprising a locking surface, the locking recesses and associated locking surfaces configured to receive a complementary locking mechanism having a plurality of arms with each arm having a locking barb, and the recesses being spaced apart from each other so that the arms of the locking mechanism must resiliently bend inwardly to be introduced into and withdrawn from the recesses, and each arm having a barb to engage a locking surface of the respective recess in which the arm is placed to thereby temporarily lock the mechanism into a fixed position in relation to the associated injection site.

13. The injection port of claim 1, further comprising a locking mechanism configured to be mounted at a distal end of a delivery device to engage the locking features of the body, wherein:
the locking features comprise a plurality of recesses formed in the body on either side of an associated injection site, each recess comprising an inner locking surface;
the locking mechanism comprising a plurality of resiliently bendable locking arms with each arm having a locking barb formed for engaging a locking surface, each arm being resiliently bendable inwardly to be introduced into and withdrawn from a recess, and each arm having a normal configuration at which the barb engages the locking surface of the respective recess in which the arm is placed to thereby temporarily lock the mechanism into a fixed position in relation to the associated injection site; and
each arm having an associated bend tab configured and located so that pressing the tabs toward each other causes the arms to bend inwardly to a position at which they may be introduced into or withdrawn from the recesses.

14. An injection port for delivery of medication through skin, the port comprising:
a body having a top and a bottom surface;
an outlet cannula extending outwardly from the bottom surface of the body, the outlet cannula adapted to puncture skin of a patient, the outlet cannula having an inner surface that defines a cannula passageway through which medication is delivered;
first and second injection sites formed in the body, each of which includes a channel connecting with the cannula passageway and each of which is configured to temporarily receive medication delivery devices multiple times for delivery of medication through the cannula passageway;
wherein each injection site includes a stationary seal that is configured to be pierceable by a sharp cannula of a delivery device when such delivery device is received by the respective injection site and each of which reseals itself and its respective injection site upon removal of the sharp cannula from the seal and the delivery device from the injection site;
wherein the second injection site further includes a locking feature configured to temporarily engage a complementary locking mechanism on a delivery device to temporarily secure the delivery device in position at the second injection site for extended delivery of medication through the second injection site until the locking mechanism is disengaged from the locking feature of the second injection site and the delivery device is removed therefrom;
wherein the locking feature is configured so as to not interfere with the receipt of delivery devices at the second injection site that can be received at the first injection site.

15. The injection port of claim 14, wherein the a first injection site is located at a position that is ninety degrees from a second injection site.

16. The injection port of claim 15, wherein the first injection site is located at the top surface of the injection port and the second injection site is located at a side surface of the injection port.

17. The injection port of claim 14, wherein the first injection site comprises a first stop configured to limit a length of a delivery device that is received within the first injection site.

18. A method of injecting a patient with medication comprising:
inserting a sealed injection port through the skin of a patient and attaching the inserted injection port to the skin to keep it in place for an extended period;
temporarily engaging the injection port at a first sealed injection site thereon with a first delivery device to deliver medication to the patient through the first sealed injection port;
removing the first delivery device after delivery of the medication and resealing the first injection site;
temporarily engaging the injection port at a second sealed injection site thereon with a second delivery device to deliver medication to the patient through the second sealed injection port from a pump;
locking the second delivery device to the second injection port so that the second delivery device will remain securely in operative position at the second injection site of the port for an extended period;
unlocking the second delivery device from the second injection port and removing the second delivery device after delivery of the medication, and resealing the second injection site;
wherein the steps of engaging, delivering, removing, and resealing may be performed multiple times over the extended period that the injection port is in place.

19. The method of injecting of claim 18, wherein the step of engaging comprises piercing a stationary seal with a sharp cannula through which medication is delivered.

20. The method of injecting of claim 18, wherein the step of locking comprises:
bending a pair of locking arms toward each other;
inserting the bent locking arms into recesses formed in the injection port;
once within the recesses, allowing the arms to unbend in the recesses during which each arm engages a locking surface in the recess and resists pulling the arm from the recess thereby locking a delivery device to the injection port; and
after delivery of the medication, bending the arms inward to release the arms from the locking surfaces, and pulling the arms from the recesses to unlock them.
